(19) United States
(12) Patent Application Publication
(10) Pub. No.: US 2026/0041771 A1
(43) Pub. Date: Feb. 12, 2026

(54) NOVEL HYDRAZONE-BASED VISIBLE LIGHT-ACTIVATED NITROXYL AND NITROUS OXIDE DONORS

(71) Applicant: Trustees of Dartmouth College, Hanover, NH (US)

(72) Inventors: Ivan Abrahamian, Hanover, NH (US); Shefali Patra, Hanover, NH (US)

(73) Assignee: Trustees of Dartmouth College, Hanover, NH (US)

(21) Appl. No.: 19/293,424

(22) Filed: Aug. 7, 2025

Related U.S. Application Data
(60) Provisional application No. 63/681,026, filed on Aug. 8, 2024.

(51) Int. Cl. A61K 41/00; A61N 5/06; C07D 273/00

(52) U.S. Cl. CPC A61K 41/0042; A61N 5/062; C07D 273/00

(57) ABSTRACT

The present disclosure pertains to a molecule that is operable to release a nitroxyl (HNO) upon irradiation. The nitroxyl may be operable to convert to nitrous oxide (N₂O). The present disclosure also pertains to methods of releasing nitroxyl from a molecule of the present disclosure by irradiating the molecule. The released nitroxyl may then convert to nitrous oxide. The present disclosure also pertains to methods of administering at least one of nitroxyl or nitrous oxide to a subject by (1) administering a molecule of the present disclosure to the subject; and (2) irradiating the molecule, where the irradiation results in the release of nitroxyl from the molecule. The released nitroxyl may then convert to nitrous oxide.

### CROSS-REFERENCE TO RELATED APPLICATIONS

This application claims priority to U.S. Provisional Patent Application No. 63/681,026, filed on Aug. 8, 2024. The entirety of the aforementioned application is incorporated herein by reference.

### BACKGROUND

A need exists for more effective compounds and methods for the release of functional groups from molecules. Numerous embodiments of the present disclosure aim to address the aforementioned need.

### SUMMARY

In some embodiments, the present disclosure pertains to a molecule with the following structure:

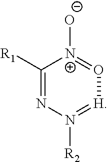

In some embodiments, a nitroxyl (HNO) is releasable from the molecule upon irradiation. In some embodiments, the nitroxyl is operable to convert to nitrous oxide (N₂O).

Additional embodiments of the present disclosure pertain to methods of releasing nitroxyl from a molecule of the present disclosure by irradiating the molecule. In some embodiments, the released nitroxyl converts to nitrous oxide. As such, in some embodiments, the methods of the present disclosure also pertain to methods of releasing nitrous oxide from the molecule.

Additional embodiments of the present disclosure pertain to methods of administering at least one of nitroxyl or nitrous oxide to a subject by (1) administering a molecule of the present disclosure to the subject; and (2) irradiating the molecule, where the irradiation results in the release of nitroxyl from the molecule. In some embodiments, the released nitroxyl converts to nitrous oxide. In some of such embodiments, the methods of the present disclosure include the administration of nitrous oxide to a subject. In some embodiments, the released nitroxyl partially converts to nitrous oxide. In some of such embodiments, the methods of the present disclosure include the co-administration of nitroxyl and nitrous oxide to a subject. In some embodiments, the released nitroxyl does not convert to nitrous oxide. In some of such embodiments, the methods of the present disclosure include the administration of nitroxyl to a subject.

### BRIEF DESCRIPTION OF DRAWINGS

FIG. 1 illustrates a process of nitroxyl (HNO) release upon light activation.

FIG. 2 provides solution-state Fourier transform infrared spectroscopy (FTIR) spectra of compound 1 (shown in FIG. 1) upon irradiation with 442 nm light at different time points (inset: characteristic N₂O peak at 2222 cm⁻¹).

FIG. 3 provides 1H nuclear magnetic resonance (NMR) spectra of 1 in CD₂Cl₂ upon irradiation with 442 nm light at different time points and comparison with the ¹H NMR spectrum of compound 2 (bottom panel, also shown in FIG. 1). The characteristic NH peak of the hydrazone 1 is highlighted.

FIGS. 4A-4B show photo-response of 1 to 442 nm irradiation in DCM (2.8×10⁻⁵ M) as monitored by UV-Vis spectroscopy (FIG. 4A) and kinetic plot of the decreasing absorbance at 432 nm fitted exponentially (FIG. 4B).

FIGS. 5A-5B show photoresponse of 1 to 394 nm irradiation in a 50:50 mixture of 1×PBS buffer and acetonitrile (2.4×10⁻⁵ M) as monitored by UV-Vis spectroscopy (FIG. 5A), and the kinetic plot of the decreasing absorbance at 399 nm fitted exponentially (FIG. 5B).

FIG. 6 shows a ¹H NMR spectrum of a fresh sample of 1 in CD₂Cl₂ compared with the spectrum of the same sample stored in the dark after 4 days.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Effective and localized delivery of drugs for disease treatment have numerous limitations. For instance, there is a growing need for effective new strategies for the treatment of heart failure. In particular, nitric oxide (NO) donors have found widespread use as cardiovascular therapeutics through their action as vasorelaxants. However, common NO-derived therapeutics suffer from major drawbacks, such as the need for a positive inotropic agent to increase myocardial contractility, vascular tolerance development, and deleterious reactivity with other reactive species (e.g., superoxide).

In contrast, nitroxyl (HNO) donors have been reported to have a remarkable edge over NO donors in treating heart failure. In particular, HNO can act as a vasorelaxant as well as a positive inotrope. Moreover, HNO does not develop tolerance. Furthermore, HNO is not sensitive to superoxide.

However, the extreme instability of HNO demands efficient donor compounds for in situ HNO production. To overcome the biologically incompatible features of conventional HNO donors (such as activation at highly alkaline pH), light-triggered HNO release based on photocaged or photoreactive compounds has evolved as an alternative strategy.

Several types of HNO donors are known. Such HNO donors include Angeli's salt, Piloty's acid and derivatives, diazeniumdiolates, and acyloxy nitroso derivatives. However, most of these compounds require potent stimuli, such as highly alkaline pH, oxidants, or enzymatic action to trigger HNO release. Such stringent conditions compromise their utility in physiologically and clinically relevant environments. Moreover, the HNO donor compounds might undergo undesirable side reactions. Furthermore, the rate of HNO release is often not optimal. This lack of suitable donors has severely hindered in-lab studies of HNO bioreactivity and prodrug development.

Photo-triggered drug delivery is a noninvasive, simplified, and tunable method to release bioactive molecules in vivo. This has inspired the design of a few photoactivatable HNO donors (e.g., photocaged Piloty's acid derivatives) that can produce HNO on demand under irradiation at suitable wavelengths.

However, existing photo-triggered HNO donors often require bioincompatible UV irradiation for activation, are unstable in the dark, need complicated and multistep synthetic routes, have non-optimal release rates, suffer from side reactions, or use toxic transition metals. As such, only a few such HNO donors have been developed.

In sum, a need exists for more effective compounds and methods for the release of functional groups from a molecule. Numerous embodiments of the present disclosure aim to address the aforementioned need.

### Molecules

In some embodiments, the present disclosure pertains to a molecule with the following structure:

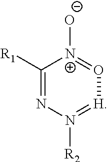

In some embodiments, each of R₁ and R₂ independently includes, without limitation, hydrogen, aromatic groups, phenyl groups, benzyl groups, aryl groups, alkyl groups, alkenyl groups, alkoxy groups, ketone groups, amine groups, amide groups, carboxyl groups, carboxylic acid groups, ester groups, thiol groups, sulfoxide groups, alcohol groups, hydroxyl groups, alkyne groups, azide groups, hydrophilic functional groups, or combinations thereof. In some embodiments, each of R₁ and R₂ independently includes, without limitation, hydrogen, aromatic groups, phenyl groups, benzyl groups, aryl groups, or combinations thereof.

In some embodiments, a nitroxyl (HNO) is releasable from the molecule upon irradiation. In some embodiments, the nitroxyl is releasable from the molecule's hydrazone moiety through a photoinduced tautomerization-driven pathway. In some embodiments, the photoinduced tautomerization-driven pathway includes a Nef reaction pathway. In some embodiments, the nitroxyl is operable to convert to nitrous oxide (N₂O).

In some embodiments, the molecule is suitable for use as a prodrug for the administration of nitroxyl and/or nitrous oxide to a subject. In some embodiments, the molecule is suitable for use in treating or preventing a disease in a subject. In some embodiments, the disease includes a cardiac condition.

The molecules of the present disclosure can include various structures. For instance, in some embodiments, the molecules of the present disclosure include the following structure:

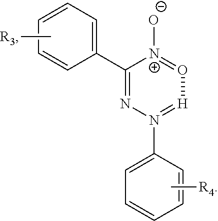

In some embodiments, each of R₃ and R₄ independently includes, without limitation, H, hydrophilic functional groups, alkenyl groups, alkoxy groups, ketone groups, amine groups, amide groups, carboxyl groups, carboxylic acid groups, ester groups, thiol groups, sulfoxide groups, alcohol groups, hydroxyl groups, alkyne groups, azide groups, or combinations thereof.

In some embodiments, the molecule includes the following structure:

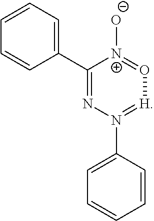

Methods of Releasing Nitroxyl from a Molecule

In additional embodiments, the present disclosure pertains to method of releasing nitroxyl from a molecule of the present disclosure. In some embodiments, the methods of the present disclosure include irradiating a molecule of the present disclosure. In some embodiments, the irradiation results in the release of nitroxyl from the molecule.

In some embodiments, the released nitroxyl converts to nitrous oxide. As such, in some embodiments, the methods of the present disclosure pertain to methods of releasing nitrous oxide from a molecule of the present disclosure.

Irradiation can occur in various manners. For instance, in some embodiments, the irradiation includes light irradiation. In some embodiments, the irradiation includes visible light irradiation. In some embodiments, the irradiation includes visible light irradiation at wavelengths of 390 nm-480 nm. In some embodiments, the irradiation includes red light irradiation. In some embodiments, the irradiation includes near infrared irradiation.

Irradiation can occur in various environments. For instance, in some embodiments, the irradiation occurs at physiological pH (i.e., a pH of 7.4).

Irradiation can be derived from various energy sources. For instance, in some embodiments, the energy source includes a light source. In some embodiments, the energy source includes a red light source.

Nitroxyl release can occur in various environments. For instance, in some embodiments, nitroxyl release can occur in vitro. In some embodiments, nitroxyl release can occur in vivo.

Methods of Administering Nitroxyl and/or Nitrous Oxide to a Subject

Additional embodiments of the present disclosure pertain to methods of administering at least one of nitroxyl or nitrous oxide to a subject. In some embodiments, the methods of the present disclosure include: (1) administering a molecule of the present disclosure to the subject; and (2) irradiating the molecule, where the irradiation results in the release of nitroxyl from the molecule.

In some embodiments, the released nitroxyl converts to nitrous oxide. In some of such embodiments, the methods of the present disclosure include the administration of nitrous oxide to a subject.

In some embodiments, the released nitroxyl partially converts to nitrous oxide. In some of such embodiments, the methods of the present disclosure include the co-administration of nitroxyl and nitrous oxide to a subject.

In some embodiments, the released nitroxyl does not convert to nitrous oxide. In some of such embodiments, the methods of the present disclosure include the administration of nitroxyl to a subject.

As set forth in more detail herein, the methods of the present disclosure may have numerous embodiments.

### Administration of Molecules

The molecules of the present disclosure may be administered to subjects in various manners. For instance, in some embodiments, the administration occurs by a method that includes, without limitation, intravenous administration, systemic administration, subcutaneous administration, transdermal administration, topical administration, intraarterial administration, intrathecal administration, intracranial administration, intraperitoneal administration, intraspinal administration, intranasal administration, intraocular administration, oral administration, intratumor administration, local administration, or combinations thereof.

In some embodiments, the administration includes systemic administration. In some embodiments, the administration includes local administration to a tissue or organ of a subject. In some embodiments, the administration includes local administration to the heart of a subject.

### Irradiation of Molecules

The molecules of the present disclosure may be irradiated in various manners. For instance, in some embodiments, the irradiation includes irradiation of a tissue or organ of a subject. In some embodiments, the irradiation includes irradiation of the heart of the subject. In some embodiments, the irradiation includes endoscopic irradiation. In some embodiments, the endoscopic irradiation includes endoscopic irradiation through the use of fiber optic cables, light-guiding catheters, or combinations thereof.

In some embodiments, the irradiation occurs after administering a molecule of the present disclosure to a subject. In some embodiments, the irradiation occurs during the administration of a molecule of the present disclosure to a subject.

In some embodiments, the irradiation includes light irradiation. In some embodiments, the irradiation includes visible light irradiation. In some embodiments, the irradiation includes visible light irradiation at wavelengths of 390 nm-480 nm. In some embodiments, the irradiation includes red light irradiation. In some embodiments, the irradiation includes near infrared irradiation.

### Energy Sources

The molecules of the present disclosure may be irradiated from various energy sources. For instance, in some embodiments, the energy source is a light source. In some embodiments, the energy source is a red light source.

### Subjects

The methods of the present disclosure may be used to administer nitroxyl and/or nitrous oxide to various subjects. For instance, in some embodiments, the subject is a human being. In some embodiments, the subject is a non-human animal.

### Treatment or Prevention of a Disease

The administration of nitroxyl and/or nitrous oxide to subjects can be used for various purposes. For instance, in some embodiments, the methods of the present disclosure may be used to treat or prevent a disease in a subject. In some embodiments, the disease includes a cardiac condition. In some embodiments, the cardiac condition includes heart failure.

More specific embodiments of the present disclosure pertain to methods of treating or preventing a cardiac condition in a subject. In some embodiments, such methods include: (1) administering a molecule of the present disclosure to the subject, where the administering results in the accumulation of the molecule in the heart of the subject; and (2) irradiating the heart, where the irradiation results in the release of nitroxyl and/or nitrous oxide into the heart of the subject.

### Additional Embodiments

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### Example 1. Development of Hydrazone-Based Visible Light-Activated Donors

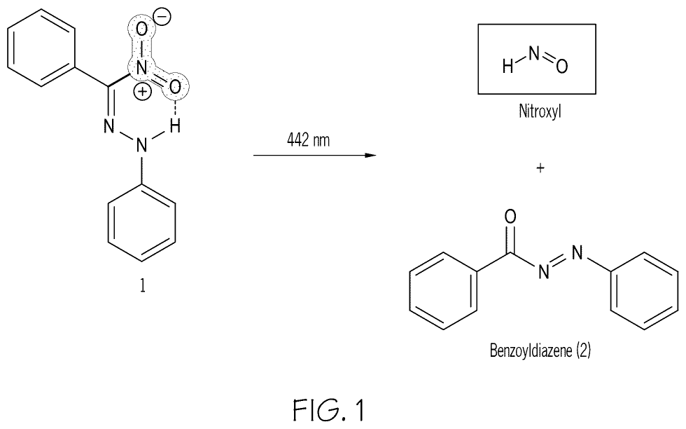

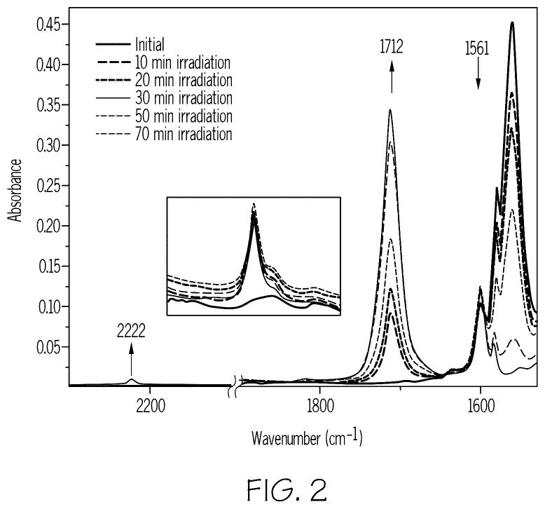

In this Example, Applicant describes the development of a novel hydrazone-based visible light-activated HNO donor (1, FIG. 1) that enables the on-demand HNO release on illumination with biocompatible visible light sources (390 to 480 nm) at physiological pH (7.4) without unwanted side reactions or instability in the dark. The detection of nitrous oxide (N₂O) in the irradiated samples using solution-state FTIR spectroscopy (FIG. 2) strongly supports the HNO release hypothesis, as N₂O is the end product of HNO degradation.

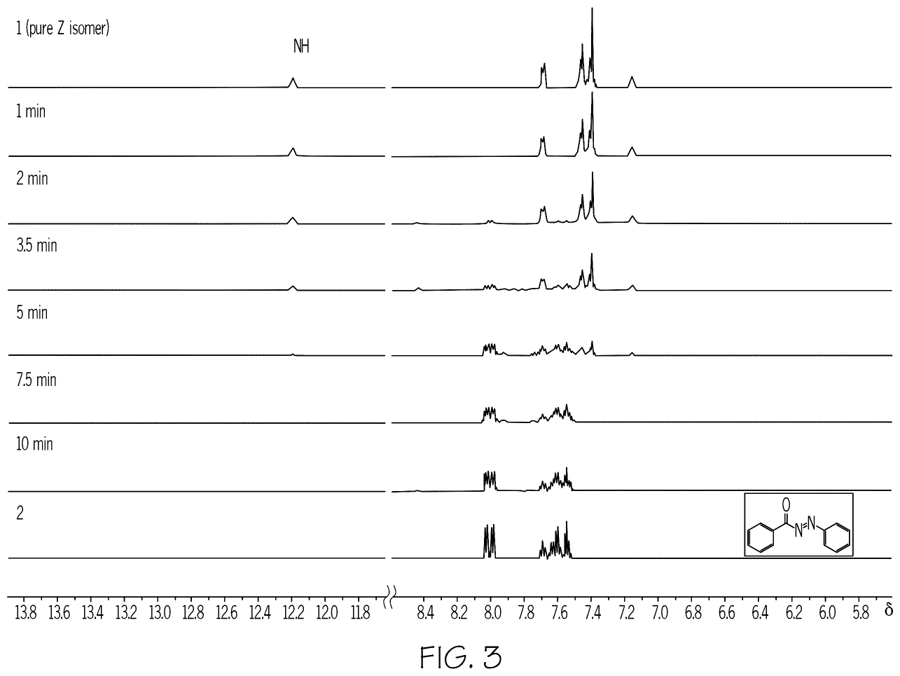

Without being bound by theory, Applicant hypothesizes that the release process relies on a new photoinduced tautomerization-driven mechanism based on the Nef reaction pathway, which is different from the mechanisms reported for other known light-activated HNO donors. ¹H NMR and UV-Vis spectroscopic studies (FIGS. 3, 4A-4B, and 5A-5B) show that the donor undergoes a clean and efficient conversion to a benzoyldiazene derivative (2, FIG. 1) without significant byproducts. Wavelengths redshifted at 480 nm can be employed to trigger the process, while maintaining optimal release rates relative to other photoactivated donors (half-lives of ˜1.5 or 9 min in dichloromethane or water-acetonitrile mixtures respectively). Accessible in two high-yielding synthetic steps (Schemes 2-3), this hydrazone potentially offers a novel photochemical pathway to produce HNO in biorelevant conditions.

### Example 1.1. Detection of N₂O as the End Product of HNO

Detecting HNO released directly is extremely challenging given its strong tendency to dimerize with other HNO molecules to form N₂O (nitrous oxide). Therefore, detecting N₂O formed in the system instead can provide strong evidence for HNO release. Applicant studied the release of N₂O in the system after photodegradation by using solution-state FTIR spectroscopy (FIG. 2).

A solution of compound 1 (FIG. 1) in DCM (2.3 mM) was loaded into a sealed IR liquid cell composed of CaF₂ windows with a 0.3 mm spacer. The cell was inserted into the cell holder of an FTIR spectrometer, and the solution of 1 sandwiched between the two CaF₂ windows was then irradiated with 442 nm light (positioned ˜1 cm from the spacer). The irradiation was off during IR data acquisition. The obtained spectra at different time points show the generation of N₂O (peak at 2222 cm⁻¹, FIG. 2) as the donor 1 (light trace) gets completely converted into the benzoyldiazene 2 (dark trace).

### Example 1.2. Identification of Compound 2 as the Byproduct

The photodegradation process was monitored by ¹H NMR spectroscopy to understand the structural changes in 1 and identify byproducts. A solution of 1 in CD₂Cl₂ was irradiated with 442 nm light and the NMR spectrum was recorded at the specified time intervals, showing its degradation into 2 (FIG. 3).

### Example 1.3. Photodegradation/Release Kinetics

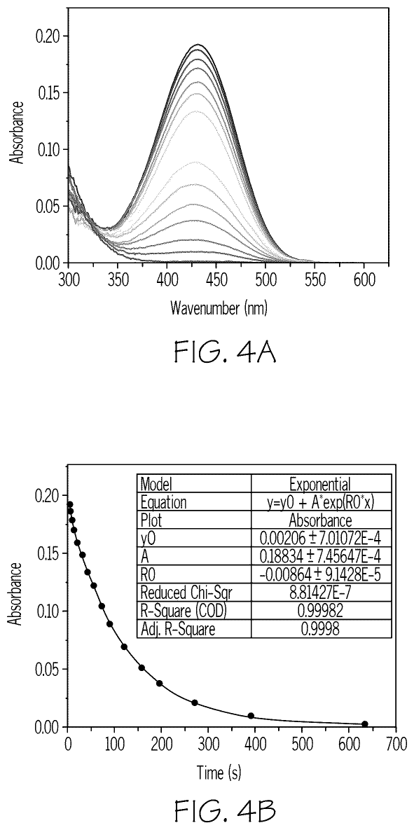

A sample of 1 in spectrophotometric DCM (2.8×10⁻⁵ M) was irradiated with 442 nm light and the changes in the UV-Vis spectra were monitored (FIGS. 4A-4B). Plotting the absorbance changes at the wavelength of maximum absorption (432 nm) against the time of illumination and performing an exponential fit shows that the half-life of the photodegradation process is 82.29±2.03 s or 1.37 min (based on three trials).

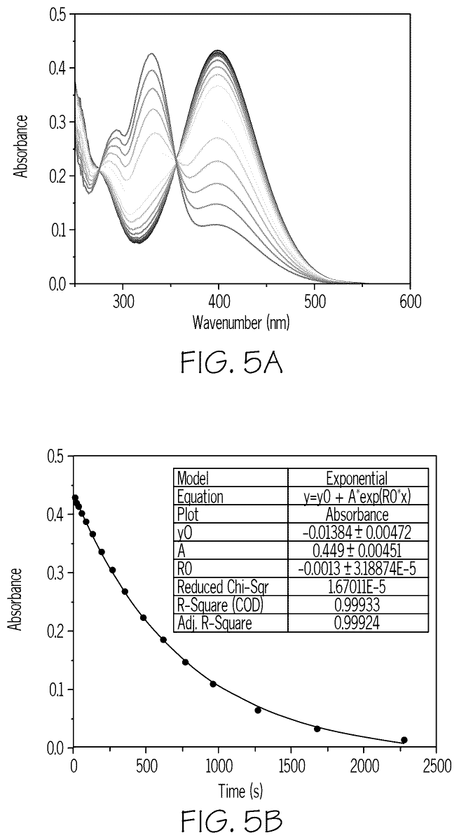

To study the behavior of the donor in physiologically relevant media, a sample of 1 in a 50:50 mixture of PBS buffer and acetonitrile (2.4×10⁻⁵ M) was irradiated with 394 nm light and changes in the UV-Vis spectra were monitored (FIGS. 5A-5B). Plotting absorbance changes at the wavelength of maximum absorption (399 nm) against the time of illumination and performing an exponential fit shows that the half-life of the photodegradation process is 8.89 min.

### Example 1.4. Stability in the Dark

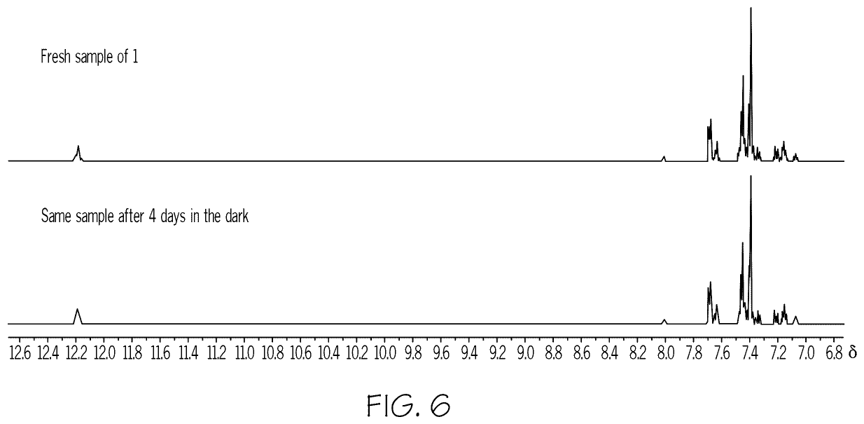

For a photoactivatable donor to be used, its stability under dark is preferable to ensure that the degradation process happens only when the compound is irradiated. A CD₂Cl₂ sample of 1 was kept in the dark and monitored for 4 days. The spectra remain unaltered, showing that this compound is stable in the dark and shows degradation only in response to light (FIG. 6).

### Example 1.5. Synthesis and Characterization

Scheme 1 illustrates the synthesis of precursor compound 3. Benzyl bromide (0.69 mL, 5.85 mmol, 1 equiv) in diethyl ether (60 mL) was added dropwise to a slurry of powdered silver nitrite (1.35 g. 8.78 mmol, 1.5 equiv) in diethyl ether (20 mL) placed in an ice bath and wrapped with aluminum foil, while maintaining the temperature below 10° C. After 3 h at 0° C., the mixture was kept at room temperature in the dark overnight. The reaction mixture was filtered on a Celite plug and washed with 50 mL diethyl ether. Sodium methoxide (348 mg, 6.44 mmol, 1.1 equiv) in methanol (10 mL) was added at 0° C. to the filtrate. The waxy precipitate was collected by filtration, washed several times with diethyl ether (50 mL), and dried under vacuum. The product was obtained as a white powder (520 mg, 56% yield). ¹H NMR (500 MHZ, D₂O) δ 7.86 (d, J=7.9 Hz, 2H), 7.45 (t, J=7.7 Hz, 2H), 7.37-7.31 (m, 1H), 7.07 (s, 1H). MS (ESI): m/z found [M+Na⁺] for C₇H₆NNa₂O₂⁺ 182.05 (calcd. 182.02).

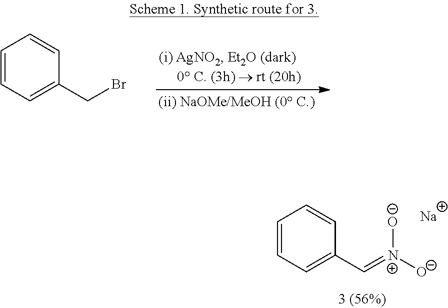 [SMILES: BrCc1ccccc1 ; [O-][N+]([O-])=Cc1ccccc1 ; [Na+]]

Scheme 2 illustrates the synthesis of compound 1. HCl (1 mL) was added dropwise to a mixture of aniline (0.09 mL, 0.94 mmol, 1.5 equiv) and water (0.5 mL) and stirred at −5 to 0° C. in a salt-ice bath for 20 min. A pre-cooled solution of sodium nitrite (65 mg, 0.94 mmol, 1.5 equiv) in 1 mL water was then added dropwise to the previous mixture over a period of 45 min. In a separate round bottomed flask, sodium acetate (258 mg, 3.14 mmol, 5 equiv) was added to a mixture of 3 (100 mg, 0.63 mmol, 1 equiv) in 5 mL EtOH/H₂O (2:1 v/v) and stirred at 0° C. for 30 min. The diazotized solution was then added dropwise to the cooled mixture containing 3. After the addition was completed, the reaction mixture was left to stir at 0° C. in dark for 30 min, and then allowed to warm up to room temperature while stirring continued for another 2 h. The reaction mixture was diluted with water (20 mL), extracted with dichloromethane (2×50 mL), and washed with brine (2×15 mL) and saturated aqueous sodium bicarbonate (2×15 mL). After drying over Na₂SO₄, the solvent was removed under reduced pressure. The residue was then subjected to silica gel column chromatography using 20:1 hexanes/ethyl acetate as eluent to give 1 (pure Z isomer) as a crystalline orange solid (152 mg, 67% yield). 1H NMR (600 MHz, CD₂Cl₂) δ 12.19 (s, 1H), 7.69 (d, J=7.7 Hz, 2H), 7.50-7.35 (m, 7H), 7.16 (t, J=6.8 Hz, 1H). ¹³C NMR (151 MHZ, CD₂Cl₂) δ 142.10, 135.59, 131.76, 131.16, 130.30, 130.03, 129.96, 129.95, 129.66, 129.40, 128.62, 125.14, 124.04, 115.79, 115.08. MS (ESI): m/z found [M+H⁺] for C₁₃H₁₂N₃O₂+242.1 (calcd. 242.09).

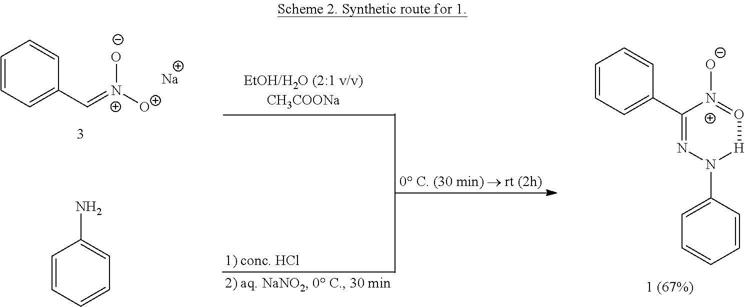

### Example 1.6. Summary

To the best of Applicant's knowledge, the aforementioned hydrazone-based compound represents the first of a novel class of visible light-activatable HNO and N₂O donating organic compounds. So far, only one other class of such compounds is known, relying on the HNO release mechanism from the conventional Piloty's acid derivatives. However, Applicant's system relies on a novel photoinduced tautomerization-driven pathway that would enhance the very few types of release mechanisms known for HNO. Furthermore, this is the first example of a visible light-driven Nef reaction without any additional reagents, which can offer a sustainable synthetic protocol for N-benzoyldiazenes and related compounds. This is also the first report of such photocleavage mechanisms in photochromic hydrazones.

As such, in contrast to existing technologies in the field of HNO, this invention adds a highly optimized class of donors with notable biocompatible features and a novel release mechanism. The hydrazone-based compounds can also produce nitrous oxide (N₂O) in a controlled manner using visible light. N₂O is an important reagent in synthetic chemistry, and is noted for its ability to selectively oxidize highly reactive species without the risk of over-oxidation. Moreover, N₂O holds great biological significance as an anesthetic and an analgesic. To address aqueous solubility issues, the hydrazone compounds can be modified by incorporating hydrophilic functional groups into the hydrazone.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

1. A molecule comprising the following structure: 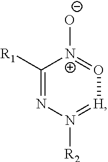
wherein each of R₁ and R₂ is independently selected from the group consisting of hydrogen, aromatic groups, phenyl groups, benzyl groups, aryl groups, alkyl groups, alkenyl groups, alkoxy groups, ketone groups, amine groups, amide groups, carboxyl groups, carboxylic acid groups, ester groups, thiol groups, sulfoxide groups, alcohol groups, hydroxyl groups, alkyne groups, azide groups, hydrophilic functional groups, or combinations thereof, and
wherein a nitroxyl (HNO) is releasable from the molecule upon irradiation.

2. The molecule of claim 1, wherein the molecule comprises the following structure: 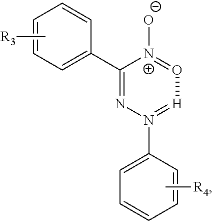
wherein each of R₃ and R₄ is independently selected from the group consisting of H, hydrophilic functional groups, alkenyl groups, alkoxy groups, ketone groups, amine groups, amide groups, carboxyl groups, carboxylic acid groups, ester groups, thiol groups, sulfoxide groups, alcohol groups, hydroxyl groups, alkyne groups, azide groups, or combinations thereof.

3. The molecule of claim 1, wherein the molecule comprises the following structure:

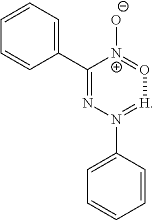

4. A method of releasing nitroxyl (HNO) from a molecule, said method comprising:
irradiating the molecule, wherein the irradiation results in the release of the nitroxyl from the molecule, and wherein the molecule comprises the following structure: 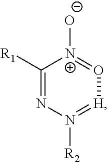
wherein each of R₁ and R₂ is independently selected from the group consisting of hydrogen, aromatic groups, phenyl groups, benzyl groups, aryl groups, alkyl groups, alkenyl groups, alkoxy groups, ketone groups, amine groups, amide groups, carboxyl groups, carboxylic acid groups, ester groups, thiol groups, sulfoxide groups, alcohol groups, hydroxyl groups, alkyne groups, azide groups, hydrophilic functional groups, or combinations thereof.

5. The method of claim 4, wherein the molecule comprises the following structure: 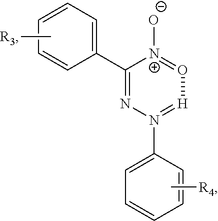
wherein each of R₃ and R₄ is independently selected from the group consisting of H, hydrophilic functional groups, alkenyl groups, alkoxy groups, ketone groups, amine groups, amide groups, carboxyl groups, carboxylic acid groups, ester groups, thiol groups, sulfoxide groups, alcohol groups, hydroxyl groups, alkyne groups, azide groups, or combinations thereof.

6. The method of claim 4, wherein the molecule comprises the following structure:

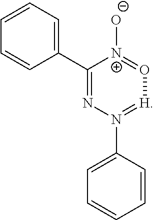

7. The method of claim 4, wherein the irradiation comprises light irradiation, visible light irradiation, red light irradiation, near infrared irradiation, or combinations thereof.

8. The method of claim 4, wherein the irradiation comprises visible light irradiation at wavelengths of 390 nm-480 nm.

9. The method of claim 4, wherein the released nitroxyl converts to nitrous oxide (N₂O), and wherein the method further comprises releasing nitrous oxide from the molecule.

10. The method of claim 4, wherein the method occurs in vitro.

11. The method of claim 4, wherein the method occurs in vivo.

12. A method of administering at least one of nitroxyl (HNO) or nitrous oxide (N₂O) to a subject, said method comprising:
administering a molecule to the subject, wherein the molecule comprises the following structure: 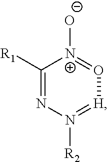
wherein each of R₁ and R₂ is independently selected from the group consisting of hydrogen, aromatic groups, phenyl groups, benzyl groups, aryl groups, alkyl groups, alkenyl groups, alkoxy groups, ketone groups, amine groups, amide groups, carboxyl groups, carboxylic acid groups, ester groups, thiol groups, sulfoxide groups, alcohol groups, hydroxyl groups, alkyne groups, azide groups, hydrophilic functional groups, or combinations thereof; and
irradiating the molecule, wherein the irradiation results in the release of the nitroxyl from the molecule.

13. The method of claim 12, wherein the released nitroxyl converts to nitrous oxide, and wherein the method comprises the administration of the nitrous oxide to the subject.

14. The method of claim 12, wherein the released nitroxyl partially converts to nitrous oxide, and wherein the method comprises the co-administration of the nitroxyl and nitrous oxide to the subject.

15. The method of claim 12, wherein the released nitroxyl does not convert to nitrous oxide, and wherein the method comprises the administration of the nitroxyl to the subject.

16. The method of claim 12, wherein the molecule comprises the following structure: 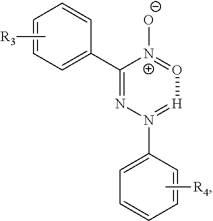
wherein each of R₃ and R₄ is independently selected from the group consisting of H, hydrophilic functional groups, alkenyl groups, alkoxy groups, ketone groups, amine groups, amide groups, carboxyl groups, carboxylic acid groups, ester groups, thiol groups, sulfoxide groups, alcohol groups, hydroxyl groups, alkyne groups, azide groups, or combinations thereof.

17. The method of claim 12, wherein the molecule comprises the following structure:

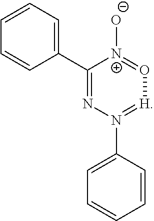

18. The method of claim 12, wherein the administration comprises systemic administration.

19. The method of claim 12, wherein the administration comprises local administration to a tissue or organ of the subject.

20. The method of claim 12, wherein the administration comprises local administration to the heart of the subject.

21. The method of claim 12, wherein the irradiation comprises irradiation of a tissue or organ of the subject.

22. The method of claim 12, wherein the irradiation comprises irradiation of the heart of the subject.

23. The method of claim 12, wherein the irradiation comprises endoscopic irradiation.

24. The method of claim 12, wherein the irradiation occurs after administering the molecule to the subject.

25. The method of claim 12, wherein the irradiation comprises visible light irradiation at wavelengths of 390 nm-480 nm.

26. The method of claim 12, wherein the subject is a human being.

27. The method of claim 12, wherein the method is used to treat or prevent a disease in the subject.

28. The method of claim 27, wherein the disease comprises a cardiac condition.

29. The method of claim 12, wherein the method is used to treat or prevent a cardiac condition in a subject, and wherein the method comprises:
administering the molecule to the subject, wherein the administering results in the accumulation of the molecule in the heart of the subject; and
irradiating the heart, wherein the irradiation results in the release of at least one of nitroxyl or nitrous oxide into the heart of the subject.
